(19) United States
(12) Patent Application Publication
(10) Pub. No.: US 2025/0000701 A1
(43) Pub. Date: Jan. 2, 2025

(54) RETINAL LASER DEVICE USING CONTROL OF NUMBER OF MICROPULSES AND CONTROL METHOD THEREOF

(71) Applicant: THE CATHOLIC UNIVERSITY OF KOREA INDUSTRY-ACADEMIC COOPERATION FOUNDATION, Seoul (KR)

(72) Inventor: Young Jung ROH, Seoul (KR)

(21) Appl. No.: 18/710,298

(22) PCT Filed: Dec. 9, 2022
(86) PCT No.: PCT/KR2022/020079

§ 371 Date: May 15, 2024

(30) Foreign Application Priority Data
Dec. 22, 2021 (KR) 10-2021-0185289

(51) Int. Cl. A61F 9/008

(52) U.S. Cl. CPC A61F 9/008; A61F 2009/00844; A61F 2009/00863

(57) ABSTRACT

An example of a retinal laser device using control of the number of micropulses comprises: an SRT laser beam irradiation unit for emitting an SRT laser beam configured by a plurality of micropulses toward the retina; an imaging unit for generating a plurality of retinal images by photographing the eyeball in real time; an image processing unit for processing and comparing the plurality of retinal images transmitted from the imaging unit to confirm a visual change in the retina, caused by the SRT laser beam; an information processing unit for setting the number of micropulses, on the basis of the energy of a damaging SRT laser beam emitted to the retina when a visual change in the retina occurs in an SRT irradiation spot; and a control unit for controlling the operation of the SRT laser beam irradiation unit according to the number of micropulses.

### TECHNICAL FIELD

The present invention relates to a retinal laser device using control of number of micropulses and control method thereof. More specifically, the present invention relates to a retinal laser device using control of micropulses that exhibits the therapeutic effect only on the retinal pigment epithelium (RPE) of the eyeball, and selectively treats only the retinal pigment epithelium of the eyeball by setting the appropriate energy of the SRT laser beam that does not damage surrounding tissues such as the choroid and then irradiating the set SRT laser beam, and its control method.

### BACKGROUND ART

Recently, the technology of treating a lesion by radiating beams to a body tissue and thereby changing the state of a tissue is widely applied.

In particular, a laser treatment technology is widely used for various lesions related to the eyeballs. For example, a device for treating the lesion of an anterior eyeball segment, such as keratoplasty, glaucoma treatment, or cataract surgery, is widely commercialized. In the retina field, a laser device to treat diabetic retinopathy, retinal tears or the like has been commercialized.

Such a laser treatment device radiates a laser to a target tissue to transmit energy thereto, thus leading to a change in a state of the tissue. However, if the energy is excessively transmitted to the target tissue, damage to adjacent tissues may occur. In particular, treatment of macular lesions may cause irreversible vision damage such as visual field defects (scotomas) due to thermal damage. In contrast, there is a problem that treatment is not performed properly, when sufficient energy is not transmitted to the target tissue. To overcome these problems, micropulse lasers with lower laser energy have been developed to avoid thermal damage to the macular area and show therapeutic effects, and are being applied to treat macular diseases.

In this regard, a conventional laser used to treat retinal diseases shown in (a) in FIG. 1 generally continuously irradiates a laser beam for 200 ms per cycle (1 cycle or 1 shot) and fires the laser beam. The firing interval of the laser beam may be adjusted by an operator.

However, the above-described conventional art had a problem in that thermal damage (burning phenomenon) occurred in the retina as a continuous wave was generated when the laser beam was fired and energy was continuously accumulated in the retinal tissue.

Accordingly, in order to avoid thermal damage to the retina caused by the laser, which is the problem of the conventional art, a method of irradiating with a micropulse laser that is a divided laser beam by splitting a laser beam continuously irradiated per cycle (1 cycle or 1 shot) for 200 ms into several pulsed lasers to the retina has been proposed, as shown in (b) of FIG. 1.

The above conventional art that controls an amount of energy of the micropulse includes a first method of controlling energy by controlling the power of the micropulse laser, and a second method of controlling micropulse duration. A duty cycle is a term referring to a ratio of time the laser is actually irradiated according to an irradiation command. For example, as shown in FIG. 1, the duty cycle of the conventional laser is 100% because the laser is irradiated continuously and without stopping during the irradiation command time. On the other hand, the conventional micropulse laser controls each micropulse duration (‘On’ time) during the duration of one shot, and controls an amount of energy with the duty cycle, which represents the proportion of a total as a percentage.

The therapeutic endpoint of a conventional micropulse laser is to stimulate the retinal pigment epithelium without leaving any damage to the retina and surrounding tissues, including the retinal pigment epithelium. Therefore, the mechanism of action and tissue response of the conventional micropulse laser are different from those of SRT laser, whose treatment endpoint is rejuvenation through damage to the retinal pigment epithelium. However, the commonality between the conventional micropulse lasers and the SRT is that they use chopped micropulses.

Another difference is that 532 nm, 577 nm, and 810 nm are mainly used as general micropulse wavelengths, and each micropulse duration is 10 to 20 ms. However, in the SRT laser device using a wavelength of 527 nm, a very short micropulse duration of 1.7 microseconds (μs) is used as a fixed parameter. Therefore, the SRT laser device cannot control an amount of laser energy by changing each micropulse duration. In addition, the irradiation frequency of the currently developed SRT laser device is 100 Hz or 500 Hz, and 100 to 500 micropulses are irradiated per second per cycle (1 cycle or 1 shot). Therefore, for 100 to 200 ms, which is the time commonly used per shot in clinical practice, 10 to 20 micropulses are fixedly irradiated at 100 Hz, or 50 to 100 micropulses are fixedly irradiated at 500 Hz. Therefore, to date, the method of controlling an amount of SRT laser energy has been to control only the power energy (pulse energy) among the laser parameters. Due to these limitations, the method for fining the appropriate amount of energy to selectively treat only the retinal pigment epithelium is the method in which after a series of laser beams with multiple power energies (pulse energy) are tested (test irradiation or pretreatment irradiation) on the periphery of the retina, diagnostic equipment such as fluorescein angiography or fundus photography is used to determine the appropriate SRT power energy.
(Patent Document 1) KR Patent Registered No. 10-1966906 (2019 Apr. 2.)

### DISCLOSURE

### Technical Problem

An object of the present invention to solve the above problems is to provide a retinal laser device using control of number of micropulses that selectively treat only the retinal pigment epithelium (RPE) by setting the appropriate energy of the SRT laser beam that does not damage eyeball tissues other than the retinal pigment epithelium (RPE) by controlling a number of micropulses and irradiating the set SRT laser beam to the retina, and a control method thereof.

The technical objects to be achieved by the present invention are not limited to the technical objects mentioned above, and other technical objects not mentioned may be clearly understood by those skilled in the art from the following descriptions.

### Technical Solution

In order to achieve the above object, the configuration of the invention provides a retinal laser device using control of a number of micropulses, the device comprising: an SRT laser beam irradiation unit that irradiates an SRT laser beam consisting of a plurality of micropulses toward a retina; an imaging unit that generates a plurality of retinal images by photographing the retina in real time; an image processing unit that processes and compares the plurality of retinal images transmitted from the imaging unit to confirm visible change in the retina caused by the SRT laser beam; an information processing unit that sets the number of micropulses based on energy of a damaging SRT laser beam irradiated to the retina when the visible change in the retina occurs; and a control unit that controls an operation of the SRT laser beam irradiation unit according to the number of micropulses such that a set SRT laser beam consisting of the set plurality of micropulses only to a retinal pigment epithelium (RPE) of the retina.

In an embodiment of the present invention, the SRT laser beam irradiation unit may irradiate the SRT laser beam with different energies toward the retina multiple times, the number of micropulses of the SRT laser beam irradiated multiple times may sequentially increase, the different energies of the SRT laser beams may be determined according to the number of the plurality of micropulses.

In an embodiment of the present invention, the plurality of retinal images may be images of the retina when the SRT laser beams that have different energies from each other are respectively irradiated, the imaging unit may transmit the plurality of retinal images to the image processing unit.

In an embodiment of the present invention, the image processing unit may process the plurality of retinal images and then compare the plurality of retinal images to confirm the visible change in the retina caused by the SRT laser beams having different energies from each other. The image processing unit may transmit energy information about the energy of a damaging SRT laser beam that causes change in the spot irradiated on the retina and the number of damaging micropulses constituting the energy of the damaging SRT laser beam to the information processing unit.

In an embodiment of the present invention, the information processing unit may set the number of therapeutic micropulses for selectively irradiating only the retinal pigment epithelium (RPE) to have a range of the number of damaging micropulses×20%≤the number of therapeutic micropulses≤the number of damaging micropulses×30%. The energy of the therapeutic SRT laser beam consisting of the plurality of therapeutic micropulses may be determined according to the number of therapeutic micropulses.

In an embodiment of the present invention, the plurality of micropulses may be set to have the same wavelength, spot diameter, and pulse duration.

In an embodiment of the present invention, the wavelength of the plurality of micropulses may be 527 nm.

In an embodiment of the present invention, the spot diameter of the plurality of micropulses may be 200 μm.

In an embodiment of the present invention, the pulse duration of the plurality of micropulses may be 1.7 us. In an embodiment of the present invention, a frequency of the SRT laser beam may be 100 hz or 500 hz.

When the frequency of the SRT laser beam is 100 Hz and the SRT laser beam is irradiated to the retina for 0.1 seconds (100 ms), the plurality of micropulses may be irradiated such that 10 micropulses are irradiated to the eyeball per cycle (1 cycle or 1 shot).

In an embodiment of the present invention, when the frequency of the SRT laser beam is 500 Hz and the SRT laser beam is irradiated to the retina for 0.1 seconds (100 ms), the plurality of micropulses may be irradiated such that 50 micropulses are irradiated to the retina per cycle (1 cycle or 1 shot).

In order to achieve the above object, the configuration of the invention provides a control method of the above described retinal laser device using control of the number of micropulses, comprising the steps of (a) irradiating, by the SRT laser beam irradiation unit, the SRT laser beam consisting of the plurality of micropulses toward the retina; (b) generating, by the imaging unit, the retina image obtained by photographing the retina in real time; (c) confirming, by the image processing unit, the visible change in the retina due to the SRT laser beam in the retina image; (d) setting, by the information processing unit, the number of therapeutic micropulses based on the energy of the damaging SRT laser beam irradiated to the retina when the visible change occurs in the retina; and (e) controlling, by the control unit, an operation of the SRT laser beam irradiation unit according to the number of therapeutic micropulses to irradiate a set therapeutic SRT laser beam only to the retinal pigment epithelium (RPE) of the retina.

### Advantageous Effects

The effect of the present invention according to the above configuration is that only the retinal pigment epithelium (RPE) is selectively treated by setting the appropriate energy of the SRT laser beam that does not damage retina tissues other than the retinal pigment epithelium (RPE) by controlling a number of micropulses and irradiating the set SRT laser beam to the retina.

The effects of the disclosure are not limited to the above-mentioned effects, and it should be understood that the effects of the disclosure include all effects that could be inferred from the configuration of the invention described in the detailed description of the invention or the appended claims.

### DESCRIPTION OF DRAWINGS

FIGS. 1(a) and (b) are graphs showing a method of determining appropriate energy by controlling the power of micropulses according to a conventional art.

FIG. 2(a) is a graph showing a change over time in the optoacoustic pressure gradient output from a device that determines appropriate energy by measuring the microbubbles generated when a SRP laser beam is absorbed by a retinal pigment epithelium (RPE).

FIG. 2(b) is a graph showing a change over time in the back-scattered light reflex signal output from a device that determines appropriate energy by measuring the microbubbles generated when a SRP laser beam is absorbed by a retinal pigment epithelium (RPE).

FIG. 3 is a block diagram showing a retinal laser device using control of the number of micropulses according to an embodiment of the present invention.

FIG. 4 is a diagram showing the spectrum of an SRT laser beam with a wavelength of 527 nm applied to a retinal laser device using control of the number of micropulses according to an embodiment of the present invention.

FIGS. 5(a) and (b) are photographs that confirm the energy of a therapeutic SRT laser beam through fluorescent fundus photography.

FIGS. 6(a) and (b) are diagram showing the results of irradiating an SRT laser beam only to a retinal pigment epithelium (RPE) using a retinal laser device using control the number of micropulses according to an embodiment of the present invention and the side effects that occur when irradiating the eyeball with a conventional laser according to a conventional art.

FIGS. 7(a) and (b) are diagrams a color fundus photograph and fluorescent fundus photograph taken of the eyeball irradiated with an SRT laser beam through a retinal laser device using control of the number of micropulses according to an embodiment of the present invention.

FIG. 8 is a diagram showing a state of the eyeball according to a number proportion of micropulses.

FIGS. 9(a) and (b) are diagrams showing visible change in the eyeball according to the number of micropulses.

FIGS. 10(a) and (b) are diagrams showing the number of micropulses and diameter of spot of the micropulses for a duration of 0.1 seconds per cycle (1 cycle or 1 shot) used in a retinal laser device using control of the number of micropulses according to an embodiment of the present invention.

FIG. 11 is a flowchart showing a method of controlling a retinal laser device using control of the number of micropulses according to an embodiment of the present invention.

FIG. 12 is a flowchart showing an operation process for determining the appropriate energy of a SRT laser beam in a method of controlling a retinal laser device using control of the number of micropulses according to an embodiment of the present invention.

FIG. 13 is a flowchart showing an operation process of determining the appropriate energy of a SRT laser beam and then irradiating the therapeutic SRT laser beam to the eyeball in a method of controlling a retinal laser device using control of the number of micropulses according to an embodiment of the present invention.

FIG. 14 is a cross-sectional diagram showing a cross section of an eyeball including retinal pigment epithelium (RPE).

FIG. 15 is a cross-sectional diagram showing a retinal pigment epithelium (RPE) and the tissues of the eyeball adjacent to the retinal pigment epithelium (RPE).

FIGS. 16(a) and (b) are photographs showing a retinal pigment epithelium (RPE) immediately after and 4 days after treatment using a method of controlling a retinal laser device using control of the number of micropulses according to an embodiment of the present invention.

FIGS. 17(a) and (b) are scanning electron microscope photographs showing damage to a retinal pigment epithelium (RPE) layer in an SRT spot immediately after a procedure through a method of controlling a retinal laser device using control of the number of micropulses according to an embodiment of the present invention is performed on the retina of a rabbit, and recovery of the SRT spot with proliferation of the RPE 7 days after the procedure.

FIGS. 18(a) and (b) are photographs showing an eyeball 1 hour after treatment and 2 to 3 months after treatment using a method of controlling a retinal laser device using control of the number of micropulses according to an embodiment of the present invention.

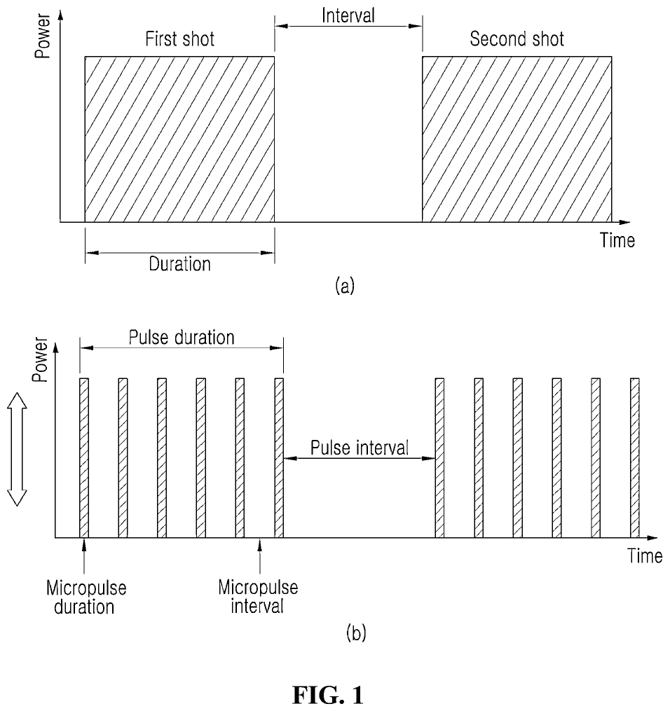

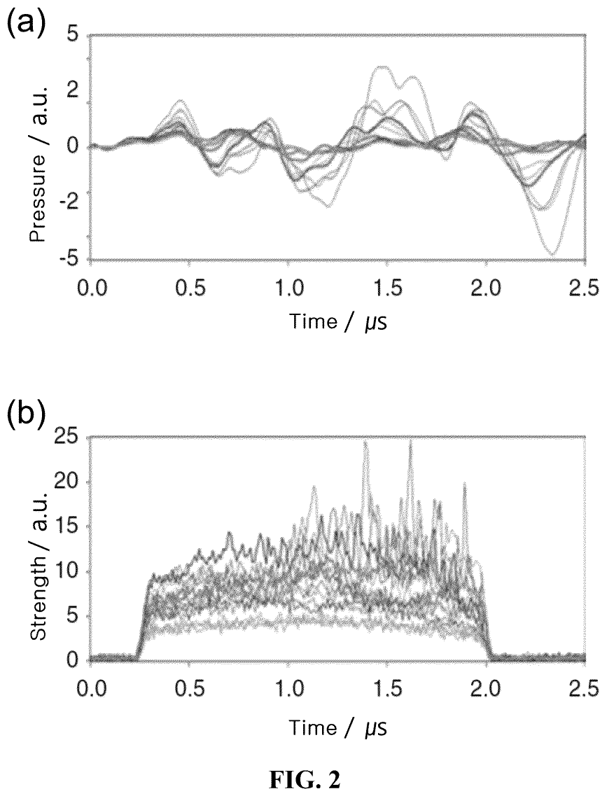

### BEST MODE FOR INVENTION

Hereinafter, The most preferable embodiment of the present invention includes an SRT laser beam irradiation unit that irradiates an SRT laser beam consisting of a plurality of micropulses toward a retina; an imaging unit that generates a plurality of eyeball images by photographing an eyeball in real time; an image processing unit that processes and compares the plurality of eyeball images transmitted from the imaging unit to confirm visible change in the eyeball caused by the SRT laser beam; an information processing unit that sets the number of micropulses based on energy of a damaging SRT laser beam irradiated to the retina when the visible change in the eyeball occurs; and a control unit that controls an operation of the SRT laser beam irradiation unit according to the number of micropulses such that a set SRT laser beam consisting of the set plurality of micropulses only to a retinal pigment epithelium (RPE) of the retina.

### MODE FOR INVENTION

Hereinafter, the disclosure will be explained with reference to the accompanying drawings. The disclosure, however, may be modified in various different ways, and should not be construed as limited to the embodiments set forth herein. Also, in order to clearly explain the disclosure, portions that are not related to the disclosure are omitted, and like reference numerals are used to refer to like elements throughout the specification.

Throughout the specification, it will be understood that when an element is referred to as being “connected (accessed, contacted, coupled) to” another element, it may include not only cases where they are “directly connected,” but also cases where they are “indirectly connected” with another member in between. Also, it will also be understood that when a component “includes” an element, unless stated otherwise, this means that the component can additionally include other elements, rather than excluding other elements.

The terms used herein are only used to describe specific embodiments and are not intended to limit the disclosure. Singular expressions include plural expressions unless the context clearly dictates otherwise. In the specification, it will be further understood that the terms “comprise” and “include” specify the presence of stated features, integers, steps, operations, elements, components, or combinations thereof, but do not preclude in advance the possibility of the presence or addition of one or more other features, integers, steps, operations, elements, components, or combinations.

Hereinafter, embodiments of the disclosure will be described in detail with reference to the accompanying drawings.

### 1. Retinal Laser Device 100 Using Control of the Number of Micropulses

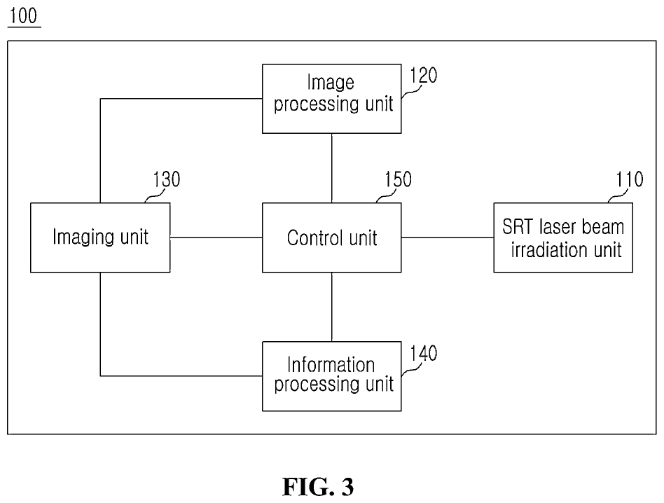

Hereinafter, a retinal laser device using control of the number of micropulses according to an embodiment of the present invention will be described with reference to FIGS. 3 to 9.

FIG. 3 is a block diagram showing a retinal laser device using control of the number of micropulses according to an embodiment of the present invention.

Referring to FIG. 3, a retinal laser device 100 using control of the number of micropulses according to an embodiment of the present invention includes an SRT laser beam irradiation unit 110, an imaging unit 120, an image processing unit 130, and an information processing unit 140 and a control unit 150.

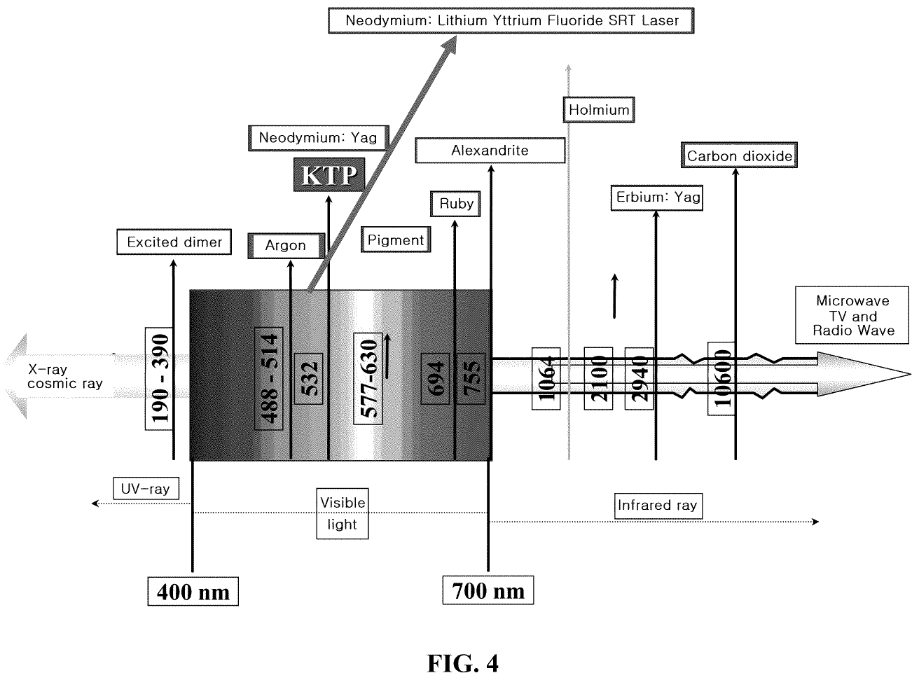

FIG. 4 is a diagram showing the spectrum of an SRT laser beam applied to a retinal laser device using control of the number of micropulses according to an embodiment of the present invention.

Referring to FIG. 4, the SRT laser beam irradiation unit 110 irradiates an SRT laser beam consisting of a plurality of micropulses toward the retina.

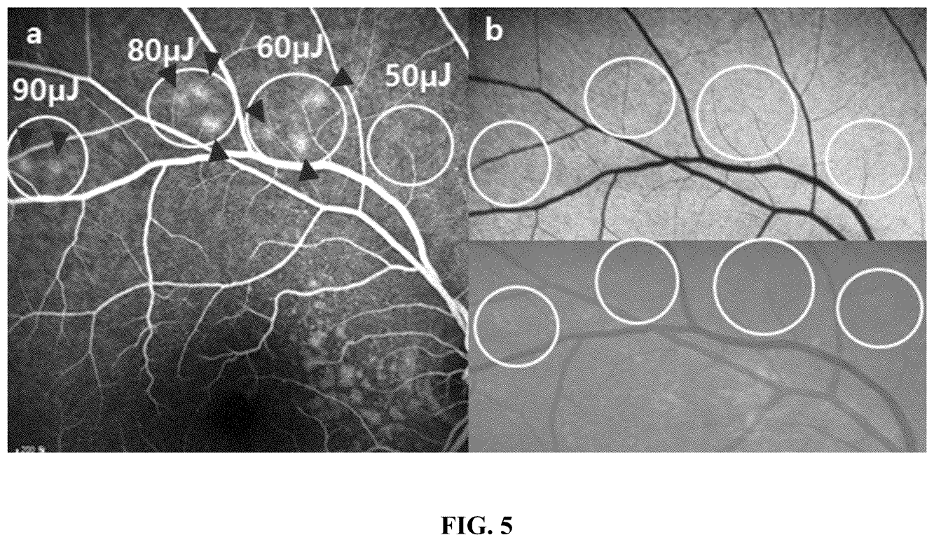

FIGS. 5(a) and (b) are photographs that confirm the energy of a therapeutic SRT laser beam through fluorescent fundus photography. The changes caused by the appropriate SRT laser beam are not visible (invisible) in color fundus photography and are only confirmed (visible) in fluorescent fundus photography.

The SRT laser beam irradiation unit 110 irradiates a test spot to the eyeball in order to determine the appropriate energy of the SRT laser beam to selectively irradiate and treat only the retinal pigment epithelium (RPE) while minimizing damage to the eyeball.

Specifically, the SRT laser beam irradiation unit 110 irradiates the SRT laser beams of different energies (50 μJ, 60 μJ, 80 μJ, 90 μJ) multiple times to increase the power energy (pulse energy) in a stepwise manner to the periphery of the retina. The SRT laser beam, which is irradiated in multiple times, is irradiated as a bundle of 10 micropulses per shot when the frequency of the laser device is 100 Hz (50 micropulses in the case of 500 Hz). In this case, if the energy at which retinal changes begin to appear in the naked eyeball or fundus photography is 90 μJ, the 90 μJ SRT laser beam controlled to the number of micropulses of 20 to 30% of 10 micropulses through the control unit is irradiated to a treatment area for treatment endpoint.

This is a method of titrating power energy (pulse energy) that selectively acts only on the RPE by using the feature that the pulse energy increases as the number of micropulses per 1 shot (or 1 cycle) of the SRT laser beam increases, and decreases as the number of micropulses per 1 shot decreases,

Since the pulse energy on the retina, which can be seen with the naked eyeball through the above process, represents slightly higher energy than the appropriate SRT energy to selectively treat RPE, the appropriate energy of the SRT laser beam at which microbubbles, which are the appropriate energy of SRT, begin to be generated, is set by controlling the number of micropulses to a lower energy than the energy. Accordingly, the SRT laser beam irradiation unit 110 irradiates the therapeutic SRT laser beam set by the control of the control unit 150 to the eyeball to selectively irradiate and treat only the retinal pigment epithelium (RPE).

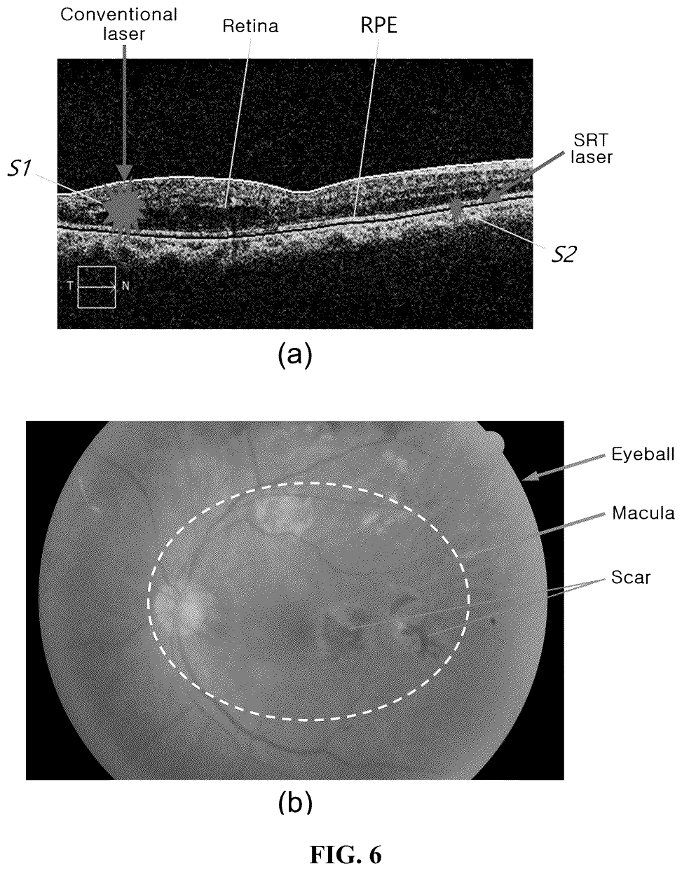

FIGS. 6(a) and (b) are diagram showing the results of irradiating an SRT laser beam only to a retinal pigment epithelium (RPE) using the retinal laser device using control the number of micropulses according to an embodiment of the present invention and the side effects that occur when irradiating the eyeball with a conventional laser according to a conventional art. Changes in the retina caused by the SRT laser beam are difficult to be observed in optical coherence tomography because they show very subtle changes only in the RPE layer. However, conventional lasers cause structural changes in the entire retina due to thermal damage, which is also observed in optical coherence tomography.

FIG. 6(a) shows an area (S1) damaged when the eyeball is irradiated with the conventional laser beam according to a conventional art and an area (S2) damaged when the eyeball is irradiated with the SRT laser beam according to the retinal laser device using control of the number of micropulses according to an embodiment of the present invention.

As shown in FIG. 6(a), when the eyeball is irradiated with the conventional laser beam according to the conventional art, the retina (S1) itself is damaged. On the other hands, when the eyeball is irradiated with the SRT laser beam by the retinal laser device using control of the number of micropulses according to an embodiment of the present invention, only the retinal pigment epithelium (RPE) (S2) is selectively damaged.

The retinal pigment epithelium (RPE) is regenerated after temporary damaged and is therefore treated by the present invention.

FIG. 6(b) shows side effects such as retinal scarring that occur when the eyeball is irradiated with the conventional laser beam according to the conventional art.

When the conventional laser beam is irradiated to the eyeball, as shown in FIG. 6(b), scars (black) appear on the retina and visual cells are destroyed, causing a side effect (scotoma) in which part of the patient's field of vision disappears during macular treatment.

In order to determine the appropriate energy of the SRT laser beam to selectively irradiate and treat only the retinal pigment epithelium (RPE) while minimizing damage to the eyeball, in the present invention in order to solve the above conventional problems, while sequentially increasing the energy of the SRT laser beam, the SRT laser beam is experimentally irradiated to the periphery of the retina until the moment microbubbles are generated in the retina. Changes in the retina that occur during the above process can be confirmed directly by the operator with the naked eyeball or through image analysis through the image processing unit 130, which processes multiple eyeball images acquired by the imaging unit 120.

The imaging unit 120 generates a plurality of eyeball images captured in real time.

In this case, the plurality of eyeball images are images of the eyeball when the SRT laser beams of different energies are respectively irradiated.

The imaging unit 120 described above transmits the plurality of eyeball images to the image processing unit 130.

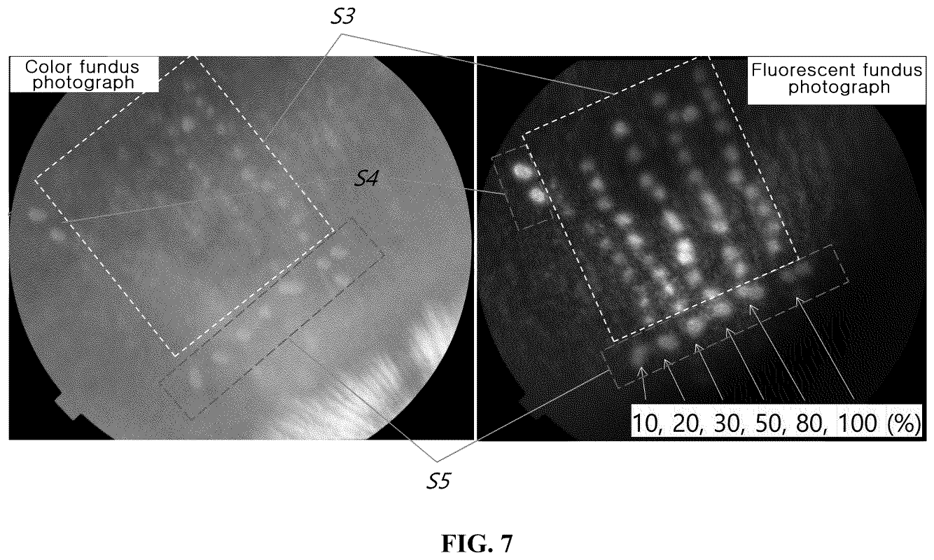

FIGS. 7(a) and (b) are diagrams showing animal experiment using rabbits with a color fundus photograph and fluorescent fundus photograph taken of the eyeball irradiated with an SRT laser beam through the retinal laser device using control of the number of micropulses according to an embodiment of the present invention. A spot in a red square is a marker burn for marking, and a spot in a yellow square represents a change in a SRT spot according to a change in the number of micropulses.

The image processing unit 130 processes and compares the plurality of eyeball images transmitted from the imaging unit 120 to confirm visible change in the eyeball due to the SRT laser beam.

Referring to FIGS. 7(a) and (b), the image processing unit 130 processes the plurality of eyeball images and then compares the plurality of eyeball images to determine visible change in the eyeball due to the SRT laser beams of different energies.

FIG. 7(a) is a color fundus photograph to confirm visible change (visible burn) in the SRT retina, and FIG. 7(b) is a fluorescent fundus photograph to confirm visible change (visible burn) in the eyeball.

Specifically, in FIGS. 7(a) and (b), a number of SRT spots are marked as a yellow box area (S3), and when 10% to 100% of the energy of the damaging SRT laser beam is irradiated to the eyeball, a number of SRT spots are marked with a red box area (S4). Using the SRT laser beam that consists of 10 micropulses and shows retinal changes as a 100% standard, the irradiation was performed while varying the number of micropulses from 1 to 10.

That is, when examining 10% to 100% of the number of micropulses through comparison of FIGS. 7 (a) and (b), when the laser is irradiated to the retina, the SRT appropriate energy is observed only in fluorescent fundus photography but not in color fundus photography in a range of 20 to 30% of the number of micropulses. When the number of micropulses is 10%, the SRT appropriate energy is not observed in either fluorescent angiography or color angiography, indicating insufficient energy to cause an SRT spot. The white laser spot in the red box area (S4) is a marker burn necessary for orientation for biopsy.

Comparing FIGS. 7(a) and (b), when 10% of the damaging SRT laser beam is irradiated to the eyeball, the SRT spot is not visible in the color fundus photograph and the fluorescent fundus photograph. When 20 to 30% of the damaging SRT laser beam is irradiated to the eyeball, the SRT spot is not visible in the color fundus photograph but is visible in the fluorescent fundus photograph.

In addition, when 50% of the damaging SRT laser beam is irradiated to the eyeball, the SRT spot is visible in both color fundus photographs and fluorescent fundus photographs, and even when 80 to 100% of the damaging SRT laser beam is irradiated to the eyeball, the SRT spot is visible in both color fundus photographs and fluorescent fundus photographs. This indicates that the energy is higher than the appropriate energy to generate an SRT spot.

Through the above, it was confirmed that the therapeutic SRT laser beam, which accounts for 20 to 30% of the energy of the damaging SRT laser beam, is an appropriate SRT that can treat the eyeball by selectively irradiating the retinal pigment epithelium (RPE).

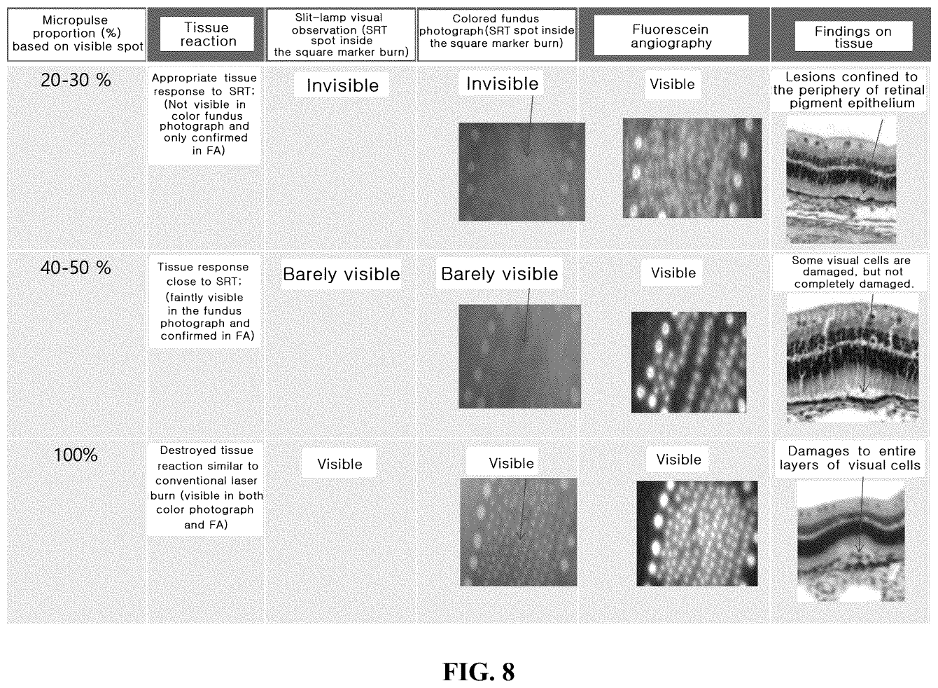

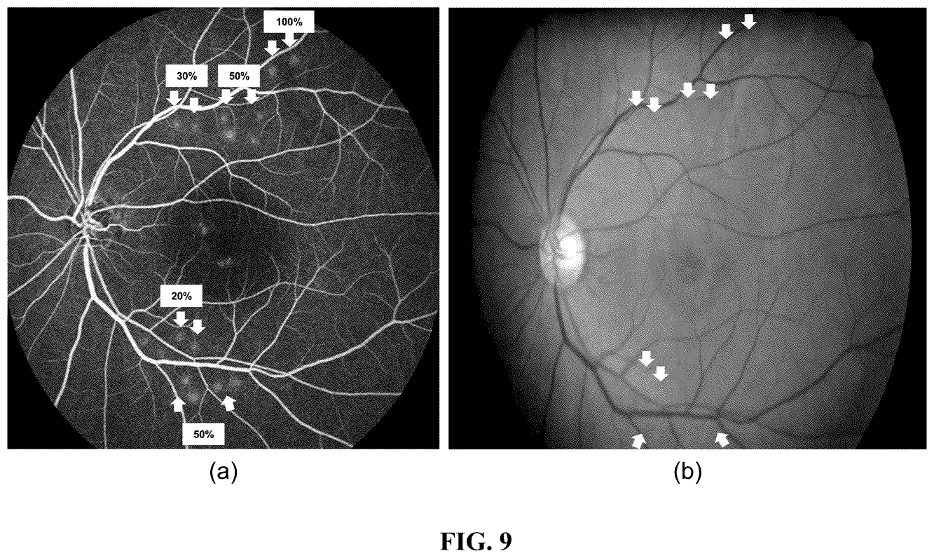

FIG. 8 is a diagram showing a state of the eyeball according to a number proportion of micropulses. FIGS. 9(a) and (b) are diagrams showing visible change in the eyeball according to the number of micropulses.

Referring to FIG. 8, when 40 to 50% of the damaging micropulses are irradiated to the eyeball, the SRT spot may be faintly confirmed in the color fundus photograph, and the SRT spot may be confirmed in the fluorescent fundus photograph. This result shows that there is some damage to visual cells, but not complete damage.

In addition, when 100% of the damaging micropulses are irradiated to the eyeball, the SRT spots may be confirmed in both color fundus photographs and fluorescent fundus photographs, and the above results show that the entire layers of visual cells are damaged.

FIG. 8 shows the appropriate energy for SRT when the number proportion of micropulses is 20 to 30%. In other words, the SRT spot is not visible in color fundus photography, but the SRT spot is observed in fluorescent angiography. Tissue reaction limited to the RPE was also observed in rabbit tissues.

On the other hand, when 20 to 30% of the damaging micropulses are irradiated to the eyeball as proposed by the present invention, the SRT spot may not be confirmed in the color fundus photograph, whereas the SRT spot may be confirmed in the fluorescent fundus photograph, and the above results are confirmed in lesions limited to the retinal pigment epithelium (RPE).

In relation to the above, FIGS. 9(a) and (b) show the state of the eyeball when 20%, 30%, 50%, and 100% of the damaging micropulses are irradiated to the eyeball. Compared to a 100% SRT spot that shows retinal changes in both color fundus photography and fluorescent fundus photography, a 20 to 30% spot is an appropriate SRT spot that is not confirmed in color fundus photography and is only confirmed in fluorescent fundus photography.

As confirmed above, the SRT spot controlled to 20 to 30% of the damaging micropulse is not observed in color fundus photography but is confirmed in fluorescent fundus photography, indicating appropriate SRT power energy (pulse energy). By irradiating appropriate SRT energy to the macula for treatment, selective treatment is possible in the RPE without damaging visual cells. For this purpose, the image processing unit 130 transmits energy information about the energy of the damaging SRT laser beam irradiated to the eyeball and the number of damaging micropulses constituting the energy of the damaging SRT laser beam to the information processing unit 140.

Meanwhile, during manual control, the operator may visually check the SRT spot on a screen monitor connected to the control unit and then manually control the SRT power energy (pulse energy).

In this case, the energy information may be the energy of the damaging SRT laser beam irradiated to the eyeball when visible change in the eyeball occurs and the number of damaging micropulses constituting the damaging SRT laser beam.

The information processing unit 140 sets the number of micropulses based on the energy of the damaging SRT laser beam irradiated to the eyeball when visible change occurs in the eyeball.

Specifically, the information processing unit 140 sets the number of therapeutic micropulses for selectively irradiating only the retinal pigment epithelium (RPE) to have a range of the number of damaging micropulses×20%≤the number of therapeutic micropulses≤the number of damaging micropulses×30%.

In this case, a plurality of micropulses is set to have the same wavelength, spot diameter, and pulse duration.

The present invention is a technology for determining appropriate energy by using the irradiation proportion of micropulses among the parameters of the SRT laser beam, and can determine the patient's appropriate energy by irradiating several test spots without applying a special device. Here, the principle that as the number of micropulses used increases, the tissue temperature of the retina increases proportionally is used.

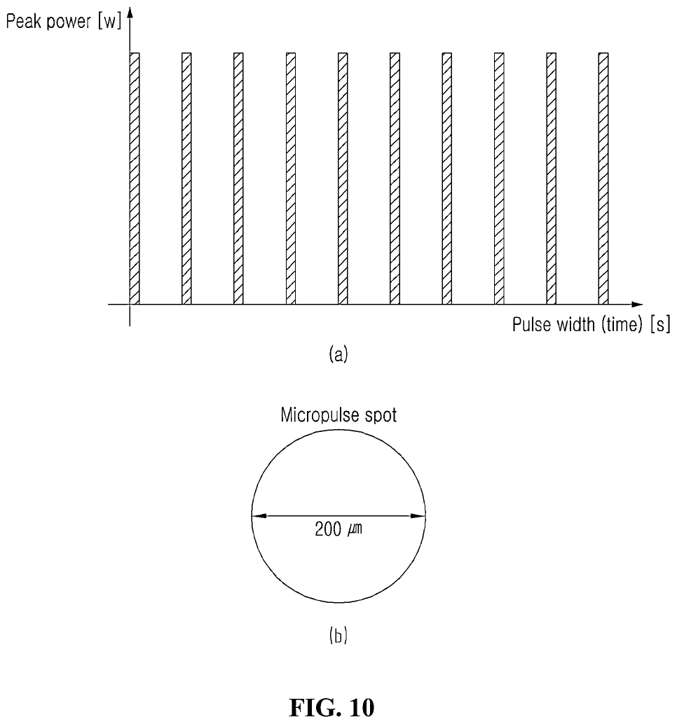

FIGS. 10(a) and (b) are diagrams showing the number of micropulses and diameter of spot of the micropulses per cycle (1 cycle or 1 shot) used in a retinal laser device using control of the number of micropulses according to an embodiment of the present invention.

FIG. 10(a) shows the peak power of micropulses according to time (Pulse width).

Specifically, as shown in FIG. 10(a), 10 micropulses have constant energy (peak power) and the same pulse interval.

Here, the wavelength of the plurality of micropulses is 527 nm.

Also, the pulse duration of the plurality of micropulses is 1.7 us.

Also, as shown in FIG. 10(b), the spot diameter of the plurality of micropulses is 200 μm.

Also, the frequency of the SRT laser beam is 100 hz or 500 hz.

Also, the power of the micropulse is irradiated to a level where visible change (visible burn) of the eyeball is faintly visible when the test spot is irradiated.

Also, in relation to the frequency of micropulses (pulse repetition rate), when the frequency of the SRT laser beam is 100 Hz, 100 micropulses are irradiated per second, but in clinical practice, treatment is performed between 0.1 and 0.2 seconds, so 10 to 20 micropulses are irradiated to the retina when a pedal is pressed once (1 cycle).

In other words, when the frequency of the SRT laser beam is 100 Hz, 20 micropulses per cycle (1 cycle or 1 shot) are irradiated to the retina during irradiation for 0.2 seconds.

However, when the frequency of the SRT laser beam is 500 Hz, 500 micropulses per second are irradiated to the eyeball, so about 100 micropulses can be irradiated to the eyeball in less than 0.2 seconds.

In other words, when the frequency of the SRT laser beam is 500 Hz, 50 to 100 micropulses per cycle (1 cycle or 1 shot) are irradiated to the eyeball during an irradiation time of 0.1 to 0.2 seconds.

In summary, the present invention irradiates the retina with an SRT consisting of 10 to 20 micropulses at 100 hz or 500 hz among the SRT laser beams as a test spot, and then sets a therapeutic SRT laser beam consisting of therapeutic micropulses corresponding to 20 to 30% of the energy of the SRT laser beam, at which visible change in the retina are confirmed, and irradiates the set SRT laser beam to the retina.

For example, when a visible spot is confirmed with 20 micropulses with a power of 150 μJ, if the therapeutic SRT laser beam reduced to 4 to 6 micropulses, equivalent to 20 to 30% of the same power of 150 μJ, is irradiated to the retina, selective damage may be caused to the retinal pigment epithelium (RPE) (when using 10 micropulses, the SRT laser beam reduced to 2 to 3 micropulses are irradiated).

Also, the energy of the therapeutic SRT laser beam consisting of a plurality of therapeutic micropulses is determined depending on the number of therapeutic micropulses.

The control unit 150 controls the operation of the SRT laser beam irradiation unit 110 according to the number of micropulses to radiate the set SRT laser beam consisting of the set plurality of micropulses only to the retinal pigment epithelium (RPE) of the eyeball.

Meanwhile, in the present invention, the technology for automatically controlling the number of micropulses has been specifically described, but manual control as follows is also possible.

Specifically, during manual control, the operator visually checks the change in the SRT laser beam within the eyeball, controls the number of micropulses of the SRT laser beam through the control unit, and then irradiates the SRT laser beam.

### 2. Control Method of a Retinal Laser Device Using Control of the Number of Micropulses

Hereinafter, a retinal laser device using control of the number of micropulses according to an embodiment of the present invention will be described with reference to FIGS. 3 to 20.

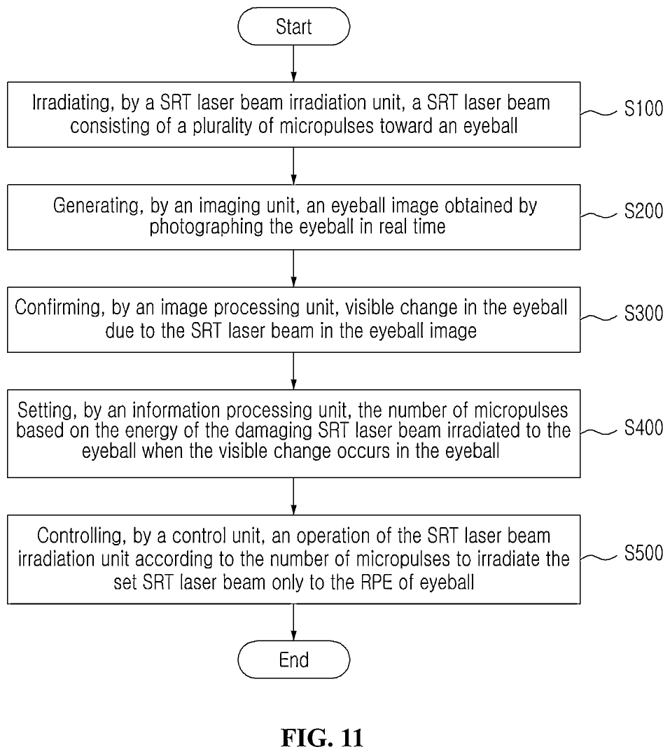

FIG. 11 is a flowchart showing a method of controlling a retinal laser device using control of the number of micropulses according to an embodiment of the present invention.

In a method of controlling a retinal laser device using control of the number of micropulses according to an embodiment of the present invention, the method of controlling the above-described retinal laser device using control of the number of micropulses includes the steps of (a) irradiating, by the SRT laser beam irradiation unit 110, the SRT laser beam consisting of the plurality of micropulses toward the retina (S100), (b) generating, by the imaging unit 120, the eyeball image obtained by photographing the retina in real time (S200), (c) confirming, by the image processing unit 130, the visible change in the retina due to the SRT laser beam in the retina image (S300); (d) setting, by the information processing unit 140, the number of therapeutic micropulses based on the energy of the damaging SRT laser beam irradiated to the retina when the visible change occurs in the retina; and (e) controlling, by the control unit 150, an operation of the SRT laser beam irradiation unit 110 according to the number of therapeutic micropulses to irradiate the set SRT laser beam only to the retinal pigment epithelium (RPE) of the retina.

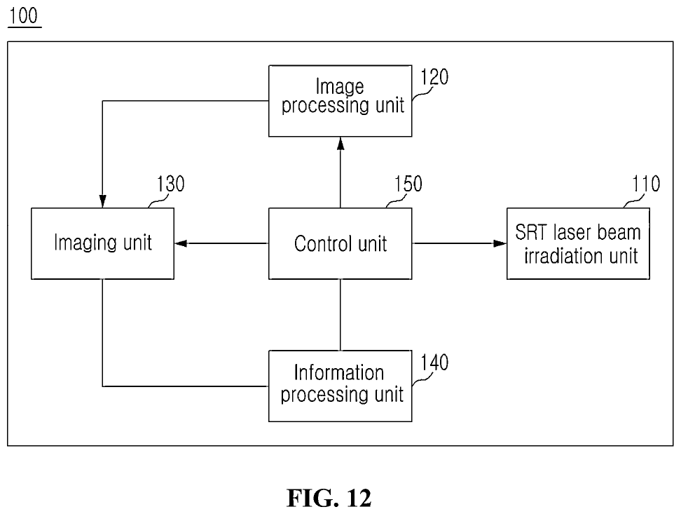

FIG. 12 is a flowchart showing an operation process for determining the appropriate energy of a SRT laser beam in a method of controlling a retinal laser device using control of the number of micropulses according to an embodiment of the present invention.

Referring to FIG. 12, in step (a), the SRT laser beam irradiation unit 110 irradiates the SRT laser beams of different energies toward the eyeball multiple times. In this case, when irradiating micropulses for 0.1 seconds and using a 100 Hz laser device, the SRT laser beam irradiated multiple times is irradiated to the retina with 10 fixed micropulses.

Here, the above process is repeatedly performed by increasing the energy (pulse energy) of the SRT laser beam only when no visible change in the eyeball occurs in the image processing unit 130 in the step (c).

Meanwhile, when a visible change in the retina occurs in the image processing unit 130 in the step (c), the SRT laser beam irradiation unit 110 irradiates the therapeutic SRT laser beam with the number of micropulses controlled according to the control of the control unit 150 in the step (e).

Next, referring to FIG. 12, in the step (b), a retinal image is generated by photographing the retina in real time, and a plurality of eyeball images by the SRT laser beams of different energies is continuously photographed and sent to the image processing unit 130.

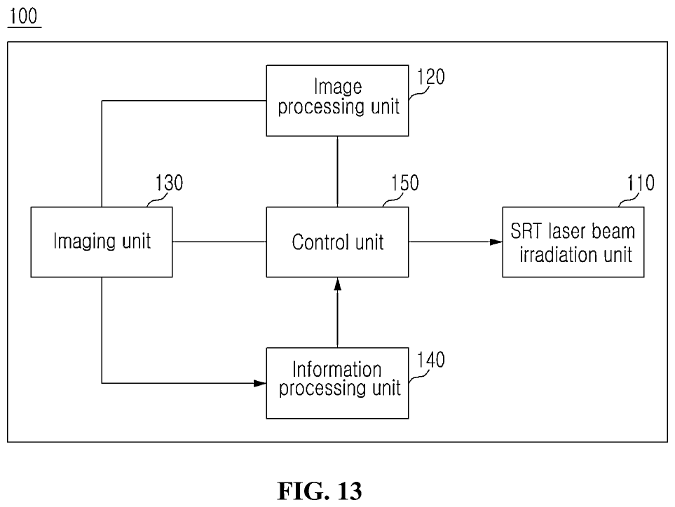

FIG. 13 is a flowchart showing an operation process of determining the appropriate energy of a SRT laser beam and then irradiating the therapeutic SRT laser beam to the retina in the method of controlling the retinal laser device using control of the number of micropulses according to an embodiment of the present invention.

Next, referring to FIG. 13, the step (c) includes the steps of (c1) comparing, by the image processing unit 130, the plurality of retina images in real time, (c2) confirming, by the image processing unit 130, the visible change in the retina due to the SRT laser beam through the comparison of the plurality of retina images; and (c3) transmitting, by the image processing unit 130, energy information about the damaging SRT laser beam irradiated to the retina to the information processing unit 140 when the visible change in the retina occurs.

In addition, the step (c) further includes the step (c4) of returning to the step (a) when the image processing unit 130 confirms no visible changes in the retina due to the SRT laser beam.

Next, referring to FIG. 13, in the step (d), the information processing unit 140 sets the number of therapeutic micropulses based on the energy of the damaging SRT laser beam irradiated to the retina when the visible change in the retina occur, In this regard, the above for detailed explanation is referred.

Lastly, in the step (e), the control unit 150 controls the operation of the SRT laser beam irradiation unit 110 according to the number of therapeutic micropulses to irradiate the set SRT laser beam only to the retinal pigment epithelium (RPE) of the retina.

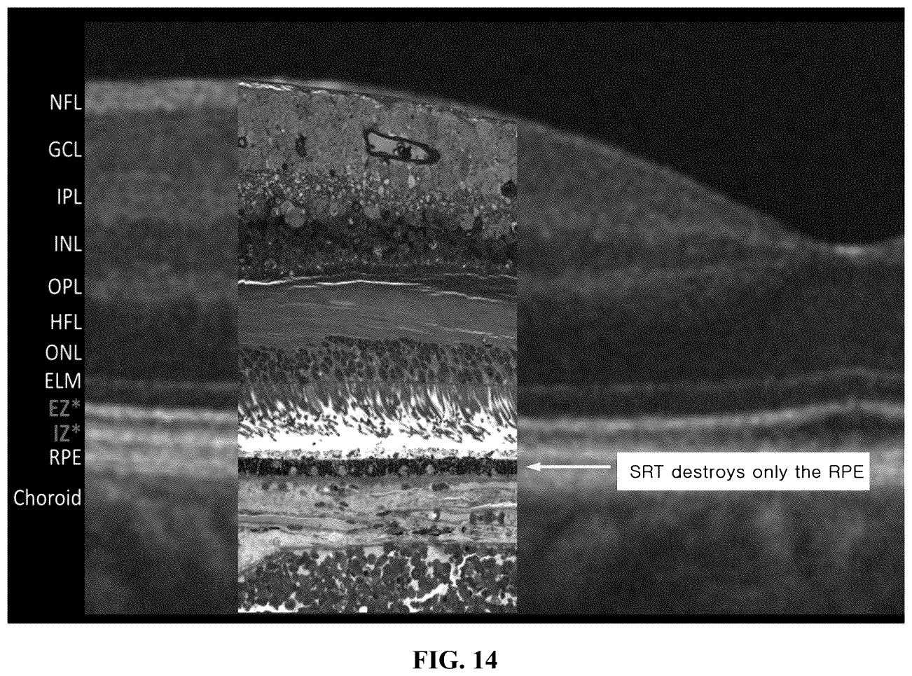

FIG. 14 is a cross-sectional diagram showing a cross section of an eyeball including retinal pigment epithelium (RPE). FIG. 14 shows the choroid, which constitutes the retina and surrounding tissues, the retinal pigment epithelium (RPE), the outer nuclear layer (ONL), inner nuclear layer (INL), inner plexiform layer (IPL), ganglion cell layer (GCL), and the like.

As shown in FIG. 14, the SRT laser beam according to the retinal laser device using control of the micropulses and control method thereof according to an embodiment of the present invention selectively destroys only the retinal pigment epithelium (RPE) and the destroyed retinal pigment epithelium (RPE) is recovered as it regenerates after a certain period of time.

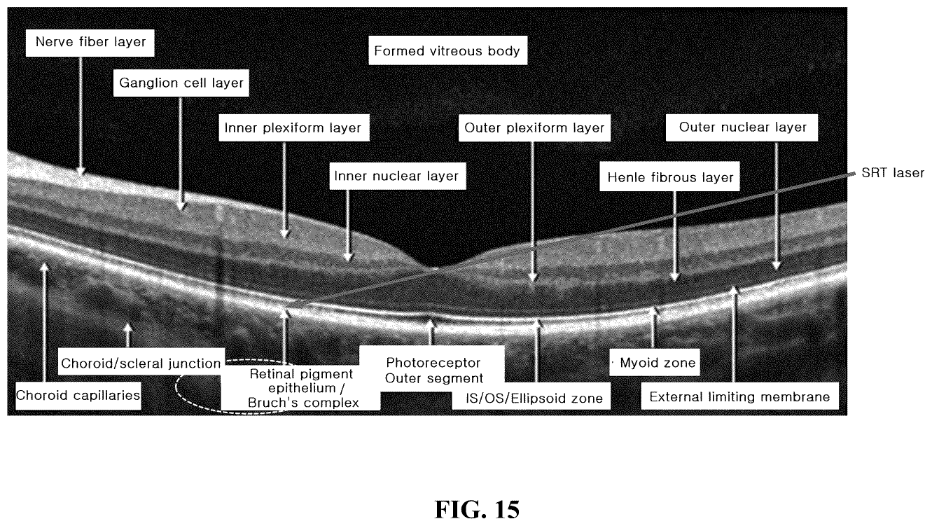

FIG. 15 is a cross-sectional diagram showing a retinal pigment epithelium (RPE) and the tissues of the retina adjacent to the retinal pigment epithelium (RPE).

As shown in FIG. 15, the SRT laser beam should be irradiated only to the retinal pigment epithelium (RPE) for treatment while preventing damage to other surrounding tissues.

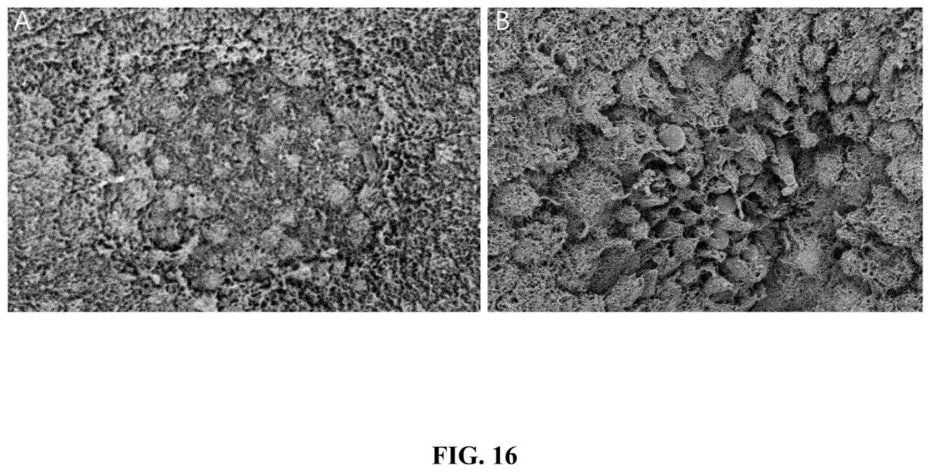

FIGS. 16(a) and (b) are photographs showing a retinal pigment epithelium (RPE) before and after treatment using a method of controlling the retinal laser device using control of the number of micropulses according to an embodiment of the present invention.

FIG. 16(a) shows the retinal pigment epithelium (RPE) immediately after irradiation with the SRT laser beam according to the present invention, and it can be seen that the retinal pigment epithelium (RPE) is damaged in the center of the SRT spot.

After 7 days of irradiation with the SRT laser beam, it can be confirmed that the damaged retinal pigment epithelium (RPE) has proliferated and the damaged area has recovered, as shown in FIG. 16(b).

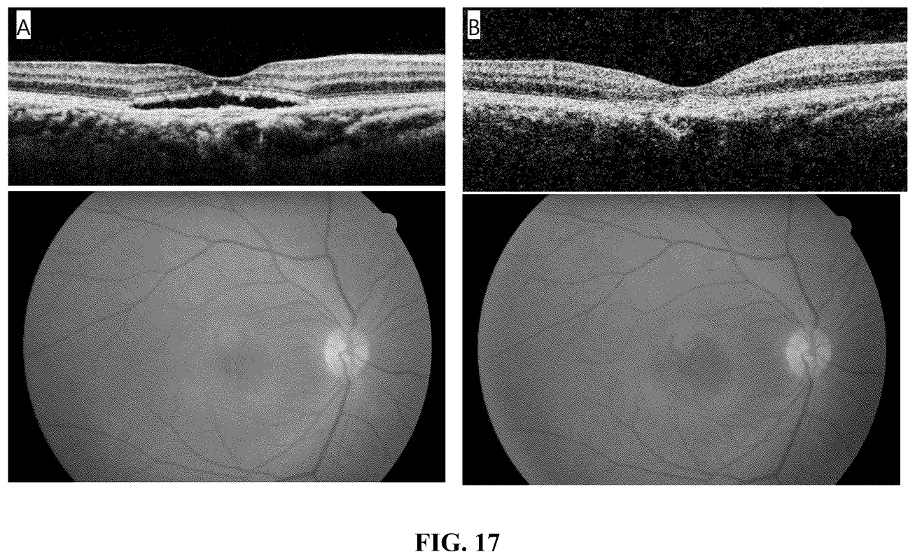

FIGS. 17(a) and (b) are photographs before and after treatment of the eyeball of a patient with central serous chorioretinopathy using a method of controlling the retinal laser device using control of the number of micropulses according to an embodiment of the present invention.

In the eyeball of the patient with central serous chorioretinopathy before the SRT treatment shown in FIG. 17(a), it can be confirmed that serous fluid is lost 1 month after SRT treatment, as shown in FIG. 17(b). The SRT spots are not observed in color fundus photography before and after the procedure.

The control method of the present invention can also be applied to other macular diseases such as dry age-related macular degeneration and diabetic macular edema.

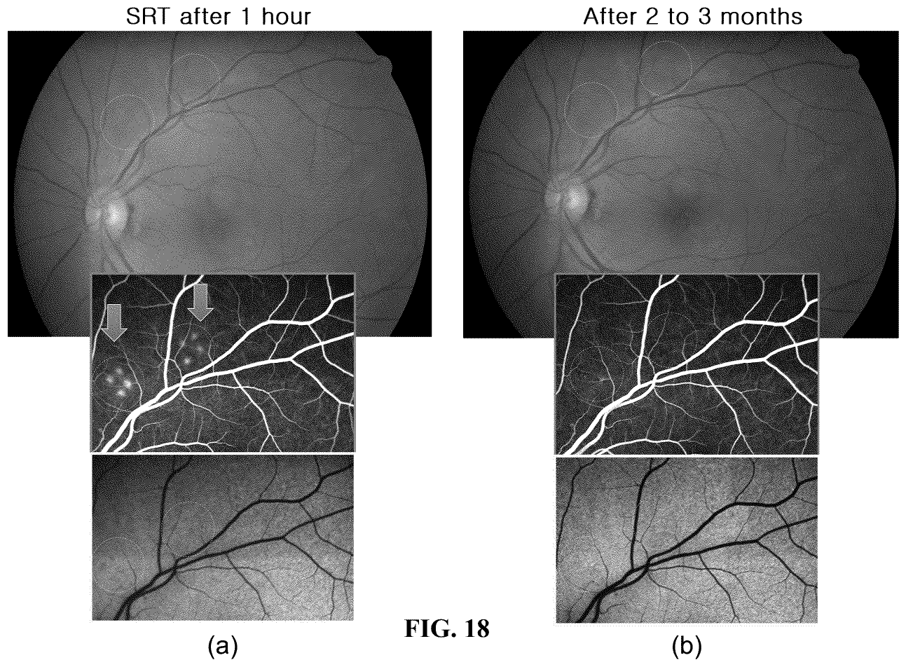

FIGS. 18(a) and (b) are fundus photographs showing the retina 1 hour after and 2 to 3 months after treating the periphery of the retina with the SRT test spot by the method of controlling the retinal laser device using control of the number of micropulses according to an embodiment of the present invention.

The appropriately controlled SRT laser beam spot is not visible to the naked eyeball, but can be confirmed through a fluorescent fundus photograph (see FIG. 18(a)) obtained through fluorescent fundus photography. In the autofluorescence photograph, which is the bottom photo, no change in the SRT spot is visible.

In addition, after 2 to 3 months after the SRT treatment, the SRT laser beam spot cannot be confirmed with the naked eyeball, fluorescent fundus photography, or autofluorescence photography due to cell regeneration of the retinal pigment epithelium (RPE) (see FIG. 18(b)).

The description of the present invention is used for illustration and those skilled in the art will understand that the present invention can be easily modified to other detailed forms without changing the technical spirit or an essential feature thereof. Therefore, the aforementioned exemplary embodiments are all illustrative in all aspects and are not limited. For example, each component described as a single type may be implemented to be distributed and similarly, components described to be distributed may also be implemented in a combined form.

The scope of the invention is to be defined by the scope of claims provided below, and all variations or modifications that can be derived from the meaning and scope of the claims as well as their equivalents are to be interpreted as being encompassed within the scope of the present invention.

### DESCRIPTION OF REFERENCE NUMERALS

100: retinal laser device using control of the number of micropulses
110: SRT laser beam irradiation unit
120: imaging unit
130: image processing unit
140: information processing unit
150: control unit

1. A retinal laser device using control of a number of micropulses,
the device comprising:
an SRT laser beam irradiation unit that irradiates an SRT laser beam including a plurality of micropulses toward a retina;
an imaging unit that generates a plurality of retinal images by photographing the retina in real time;
an image processing unit that processes and compares the plurality of retinal images transmitted from the imaging unit to confirm visible change in the retina caused by the SRT laser beam;
an information processing unit that sets the number of micropulses based on energy of a damaging SRT laser beam irradiated to the retina when the visible change in the retina occurs; and
a control unit that controls an operation of the SRT laser beam irradiation unit according to the number of micropulses to radiate a set SRT laser beam consisting of a set plurality of micropulses only to a retinal pigment epithelium (RPE) of the retina.

2. The retinal laser device of claim 1, wherein the SRT laser beam irradiation unit irradiates SRT laser beams that have different energies from each other and sequentially increase toward the retina for multiple times,
the SRT laser beams that have different energies from each other include a same number of micropulses,
among the SRT laser beams irradiated to the retina for multiple times, an SRT laser beam having energy that causes the visible change in the retina is set to 100%, and then a number of the plurality of micropulses is controlled to determine the energy of the SRT laser beams.

3. The retinal laser device of claim 2, wherein the plurality of retinal images are images of the retina when the SRT laser beams that have different energies from each other are respectively irradiated,
the imaging unit transmits the plurality of retinal images to the image processing unit.

4. The retinal laser device of claim 2, wherein the image processing unit processes the plurality of retinal images and then compares the plurality of retinal images to confirm the visible change in the retina caused by the SRT laser beams having different energies from each other,
the image processing unit transmits energy information about the energy of a damaging SRT laser beam irradiated to the retina and a number of damaging micropulses constituting the energy of the damaging SRT laser beam to the information processing unit.

5. The retinal laser device of claim 4, wherein the information processing unit sets a number of therapeutic micropulses for selectively irradiating only the retinal pigment epithelium (RPE) to have a range of the number of damaging micropulses×20%≤the number of therapeutic micropulses≤the number of damaging micropulses×30%,
energy of a therapeutic SRT laser beam including a plurality of therapeutic micropulses is determined according to the number of therapeutic micropulses.

6. The retinal laser device of claim 1, wherein the plurality of micropulses are set to have a same wavelength, spot diameter, and pulse duration.

7. The retinal laser device of claim 6, wherein the wavelength of the plurality of micropulses is 527 nm.

8. The retinal laser device of claim 6, wherein the spot diameter of the plurality of micropulses is 150 to 300 μm.

9. The retinal laser device of claim 6, wherein the pulse duration of the plurality of micropulses is 1.7 us.

10. The retinal laser device of claim 1, wherein a frequency of the SRT laser beam is 100 hz or 500 hz.

11. The retinal laser device of claim 10, wherein when the frequency of the SRT laser beam is 100 Hz, the plurality of micropulses are irradiated such that 10 to 20 micropulses are irradiated to the retina for a duration of 0.1 to 0.2 seconds per cycle (1 cycle or 1 shot).

12. The retinal laser device of claim 10, wherein when the frequency of the SRT laser beam is 500 Hz, the plurality of micropulses are irradiated such that 50 to 100 micropulses are irradiated to an eyeball for a duration of 0.1 to 0.2 seconds per cycle (1 cycle or 1 shot).

13. A control method of the retinal laser device using control of the number of micropulses of claim 1, comprising:
(a) irradiating, by the SRT laser beam irradiation unit, the SRT laser beam including the plurality of micropulses toward the retina;
(b) generating, by the imaging unit, an retinal image obtained by photographing the retina in real time;
(c) confirming, by the image processing unit, the visible change in the retina due to the SRT laser beam in the retinal image;
(d) setting, by the information processing unit, the number of therapeutic micropulses based on the energy of the damaging SRT laser beam irradiated to the retina when the visible change occurs in the retina; and
(e) controlling, by the control unit, an operation of the SRT laser beam irradiation unit according to the number of therapeutic micropulses to irradiate a set therapeutic SRT laser beam only to the retinal pigment epithelium (RPE) of the retina.
